(19) United States
(12) Patent Application Publication
(10) Pub. No.: US 2015/0297773 A1
(43) Pub. Date: Oct. 22, 2015

(54) Aroma Emitting Kit System

(71) Applicant: Denita Mayberry, Plano, TX (US)

(72) Inventor: Denita Mayberry, Plano, TX (US)

(21) Appl. No.: 14/255,038

(22) Filed: Apr. 17, 2014

(51) Int. Cl. A61L 9/03

(52) U.S. Cl. CPC A61L 9/03

(57) ABSTRACT

An aroma emitting kit system allows a person to light incense safely and easily. The system includes a housing having a striking surface positioned thereon. At least one incense device is provided. Each incense device is positionable within the housing to store the incense devices therein. Each of the incense devices includes a head and a main body coupled to the associated head. The head is ignitable and generates a flame when the head is struck against the striking surface. The main body has an incense material positioned therein. The incense material is scented and positioned adjacent to the head such that igniting the head heats the incense material and causes the incense material to emit a fragranced odor.

### BACKGROUND OF THE DISCLOSURE

### Field of the Disclosure

The disclosure relates to scent emitting systems and more particularly pertains to a new scent emitting system for allowing a person to light incense safely and easily.

### SUMMARY OF THE DISCLOSURE

An embodiment of the disclosure meets the needs presented above by generally comprising a housing having a striking surface positioned thereon. At least one incense device is provided. Each incense device is positionable within the housing to store the incense devices therein. Each of the incense devices includes a head and a main body coupled to the associated head. The head is ignitable and generates a flame when the head is struck against the striking surface. The main body has an incense material positioned therein. The incense material is scented and positioned adjacent to the head such that igniting the head heats the incense material and causes the incense material to emit a fragranced odor.

There has thus been outlined, rather broadly, the more important features of the disclosure in order that the detailed description thereof that follows may be better understood, and in order that the present contribution to the art may be better appreciated.

There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a top front side perspective view of an aroma emitting kit system according to an embodiment of the disclosure.

FIG. 2 is a side view of a housing of an embodiment of the disclosure.

FIG. 3 is a side view of an incense device of an embodiment of the disclosure.

FIG. 4 is a cross-sectional view of an embodiment of the disclosure taken along line 4-4 of FIG. 3.

FIG. 5 is an in-use view of an embodiment of the disclosure.

### DESCRIPTION OF THE PREFERRED EMBODIMENT

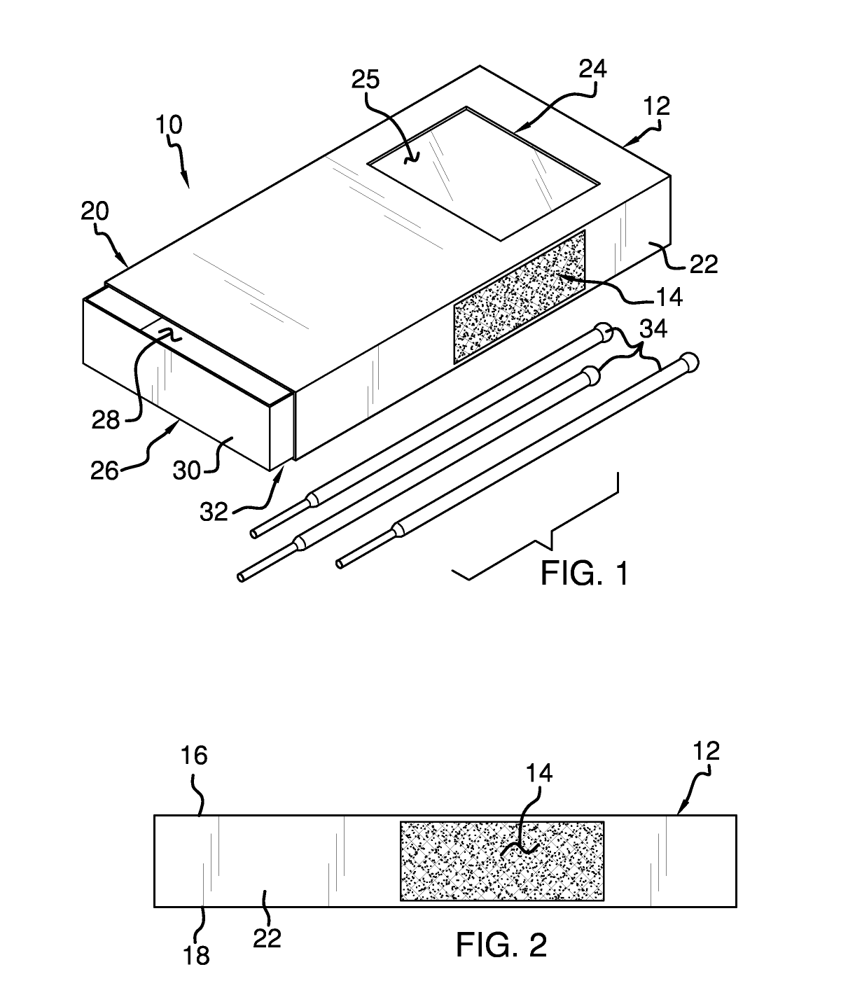

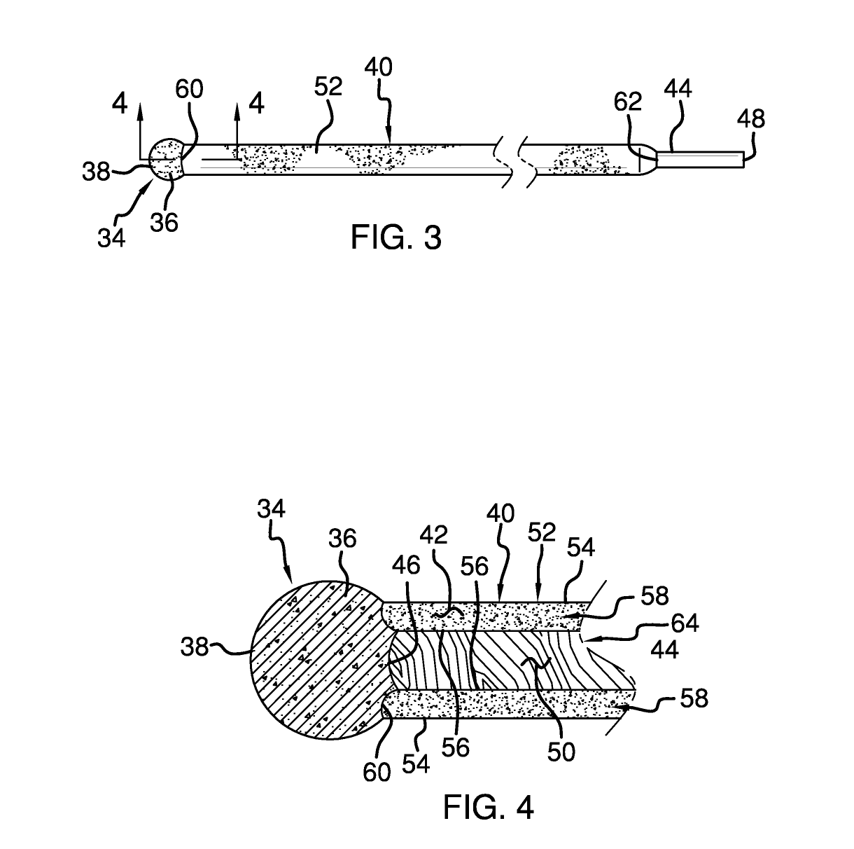

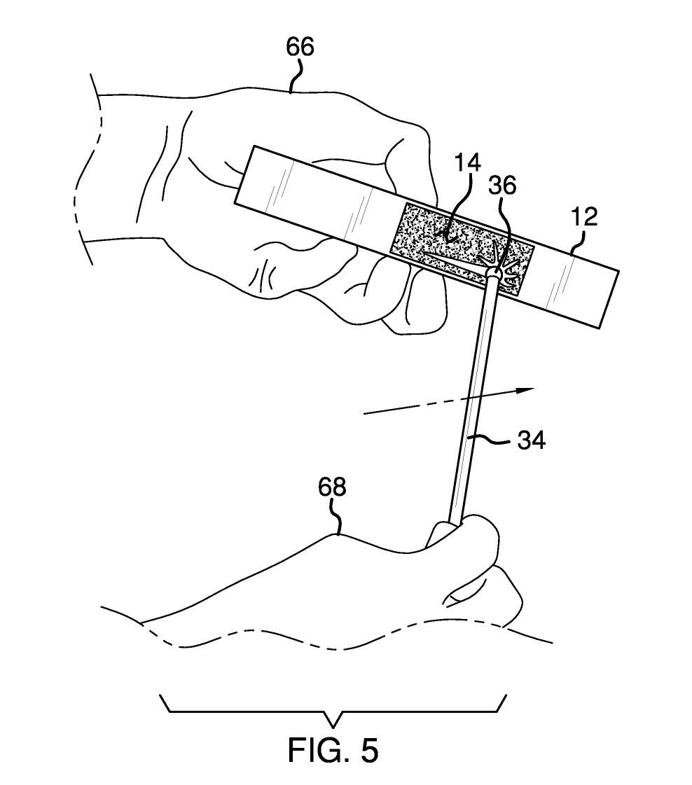

With reference now to the drawings, and in particular to FIGS. 1 through 5 thereof, a new scent emitting system embodying the principles and concepts of an embodiment of the disclosure and generally designated by the reference numeral 10 will be described.

As best illustrated in FIGS. 1 through 5, the aroma emitting kit system 10 generally comprises a housing 12 having a striking surface 14 positioned thereon. The striking surface 14 refers to a rough surface and may be of the type found on conventional matchboxes. The housing 12 comprises a top wall 16, a bottom wall 18, a first side wall 20 and a second side wall 22. The first 20 and second 22 side walls are coupled to and extend between the top wall 16 and the bottom wall 18. A window 24 with a transparent viewing panel 25 may be attached to the housing 12 to permit viewing into an interior of the housing 12 through the window 24. A tray 26 is provided having a lower wall 28 and a perimeter wall 30 coupled to and extending upwardly from the lower wall 28. The tray 26 is positionable within the housing 12 through an open end 32 of the housing 12.

At least one incense device 34 is provided. Each incense device 34 is positionable within the housing 12, and in particular, within the tray 26, to store the incense devices 34 within the housing 12. Each of the incense devices 34 includes a head 36. The head 36 comprises a generally conventional match head having a coated tip 38 that includes flammable materials, such as potassium chlorate and red phosphorus or a similar mixture of materials, such that the head 36 is ignitable and generates a flame when the head 36 is struck against the striking surface 14. The friction created by striking the head 36 against the striking surface 14 is sufficient to generate the flame. The head 36 may have a bulbous shape.

Each of the incense devices 34 includes a main body 40 coupled to the associated head 36. The main body 40 has an incense material 42 positioned therein. The incense material 42 is scented and positioned adjacent to the head 36 such that igniting the head 36 heats the incense material 42 and causes the incense material 42 to emit a fragranced odor. Each of the incense devices 34 may include a different incense material 42 each of which produces a unique scent. The main body 40 further includes an elongated stem 44 having a first end 46 and a second end 48. The first end 46 of the stem 44 is attached to the head 36. The stem 44 is comprised of a tinder substance 50, such as wood, cardboard or the like, for picking up and burning the flame generated from striking the head 36 against the striking surface 14. Each of the incense devices 34 may have a length between approximately 20.0 cm and 30.0 cm such that each incense device 34 may burn for 5 minutes or a longer period of time.

The main body 40 further includes a covering 52 having an outer surface 54 and an inner surface 56 defining a reservoir 58 therebetween. The incense material 42 is positioned within the reservoir 58. The covering 52 has a proximal end 60 and a distal end 62 with respect to the head 36. The proximal 60 and the distal 62 ends of the covering 52 may each be open. The proximal end 60 is attached to the head 36. The covering 52 has a channel 64 positioned centrally therein extending from the proximal end 60 to the distal end 62. The stem 44 is inserted into the channel 64 such that the head 36 projects outwardly from the proximal end 60. The stem 44 may project outwardly through the distal end 62 such that the second end 48 of the stem 44 extends outwardly from the channel 64.

In use, a person grasps the main body 40 with one hand 68 and the housing 12 with their other hand 66. The person's hand 68 should be spaced from the head 36 to prevent burning of the person's hand 68. The person then strikes the head 36 against the striking surface 14 to ignite the head 36 and cause the stem 44 to begin burning. Burning of the stem 44 heats the incense material 42 and causes the incense material 42 to emit a fragranced odor. The main body 40 may then be positioned in a conventional incense holder while the fragranced odor is produced.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An aroma emitting kit system comprising:
a housing having a striking surface positioned thereon; and
at least one incense device, each said incense device being positionable within said housing to store said incense devices therein, each of said incense devices including;
a head, said head being ignitable and generating a flame when said head is struck against said striking surface; and
a main body coupled to said head, said main body having an incense material positioned therein, said incense material being scented and positioned adjacent to said head such that igniting said head heats said incense material and causes said incense material to emit a fragranced odor.

2. The system of claim 1, further comprising wherein said main body includes an elongated stem having a first end and a second end, said first end of said stem being attached to said head, said stem being comprised of a tinder substance for picking up and burning the flame generated from striking said head against said striking surface.

3. The system of claim 2, further comprising wherein said main body includes a covering having an outer surface and an inner surface defining a reservoir therebetween, said incense material being positioned within said reservoir.

4. The system of claim 3, further comprising wherein said covering has a proximal end and a distal end with respect to said head, said proximal end of said covering being open, said covering having a channel positioned centrally therein extending from said proximal end to said distal end of said covering, said stem being inserted into said channel such that said head projects outwardly from said proximal end.

5. The system of claim 4, further comprising wherein said distal end of said covering is open, said stem projecting outwardly through said distal end of said covering such that said second end of said stem extends outwardly from said channel.

6. The system of claim 4, further comprising wherein said proximal end of said covering is attached to said head.

7. The system of claim 1, further comprising:
said housing comprising a top wall, a bottom wall, a first side wall and a second side wall, said first and second side walls being coupled to and extending between said top wall and said bottom wall; and
a tray having a lower wall and a perimeter wall coupled to and extending upwardly from said lower wall, said tray receiving said incense devices, said tray being positionable within said housing through an open end of said housing to store said incense devices within said housing.

8. The system of claim 1, further comprising a window having a transparent panel attached to said housing to permit viewing into an interior of said housing through said window.

9. The system of claim 1, further comprising said head having a bulbous shape.

10. An aroma emitting kit system comprising:
a housing having a striking surface positioned thereon, said housing comprising a top wall, a bottom wall, a first side wall and a second side wall, said first and second side walls being coupled to and extending between said top wall and said bottom wall;
a window having a transparent panel attached to said housing to permit viewing into an interior of said housing through said window;
at least one incense device, each said incense device being positionable within said housing to store said incense devices therein, each of said incense devices including;
a head, said head being ignitable and generating a flame when said head is struck against said striking surface, said head having a bulbous shape; and
a main body coupled to said head, said main body having an incense material positioned therein, said incense material being scented and positioned adjacent to said head such that igniting said head heats said incense material and causes said incense material to emit a fragranced odor, said main body including an elongated stem having a first end and a second end, said first end of said stem being attached to said head, said stem being comprised of a tinder substance for picking up and burning the flame generated from striking said head against said striking surface, said main body including a covering having an outer surface and an inner surface defining a reservoir therebetween, said incense material being positioned within said reservoir, said covering having a proximal end and a distal end with respect to said head, said proximal and said distal ends of said covering being open, said proximal end being attached to said head, said covering having a channel positioned centrally therein extending from said proximal end to said distal end, said stem being inserted into said channel such that said head projects outwardly from said proximal end, said stem projecting outwardly through said distal end such that said second end of said stem extends outwardly from said channel; and
a tray having a lower wall and a perimeter wall coupled to and extending upwardly from said lower wall, said tray receiving said incense devices, said tray being positionable within said housing through an open end of said housing to store said incense devices within said housing.
